(19) United States
(12) Patent Application Publication
(10) Pub. No.: US 2024/0423529 A1
(43) Pub. Date: Dec. 26, 2024

(54) OBJECTIVE DIAGNOSIS OF NEUROBEHAVIORAL DISORDERS USING EEG BIOMARKER PROCESSING

(71) Applicant: Neurizons LLC, Beverly Hills, CA (US)

(72) Inventor: Frank H. Duffy, Tunbridge, VT (US)

(73) Assignee: Neurizons LLC, Beverly Hills, CA (US)

(21) Appl. No.: 18/751,238

(22) Filed: Jun. 22, 2024

Related U.S. Application Data
(60) Provisional application No. 63/510,144, filed on Jun. 25, 2023.

(51) Int. Cl. A61B 5/372

(52) U.S. Cl. CPC A61B 5/372; G06F 17/14; G06F 18/24765; G06F 18/27; A61B 5/7203

(57) ABSTRACT

Aspects of the disclosure are directed to objective diagnosis of neurobehavioral disorders. In accordance with one aspect, the disclosure includes performing a spectral analysis on a Laplacian formatted electroencephalograph (EEG) data to generate a spectral coherence data; performing a plurality of regression analysis on the spectral coherence data to generate a smoothed spectral coherence data; performing a principal component analysis (PCA) on the smoothed spectral coherence data to generate an orthogonalized spectral coherence data; and performing a multivariate discriminant function analysis (DFA) on the orthogonalized spectral coherence data to generate a plurality of diagnostic rules and a diagnosis.

### CLAIM OF PRIORITY UNDER 35 U.S.C. § 119

The present Application for Patent claims priority to Provisional Application No. 63/510,144 entitled “OBJECTIVE DIAGNOSIS OF NEUROBEHAVIORAL DISORDERS USING EEG BIOMARKER PROCESSING” filed Jun. 25, 2023, and assigned to the assignee hereof and hereby expressly incorporated by reference herein.

### TECHNICAL FIELD

This disclosure relates generally to the field of biomedical signal processing, and, in particular, to biomedical signal processing for neurobehavioral disorder diagnosis.

### BACKGROUND

The prevalence of brain dysfunction due to neurobehavioral disorders such as autism spectrum disorder, Asperger's syndrome, attention deficit disorder, dyslexia, dementia, Alzheimer's disease, chronic fatigue syndrome, schizophrenia, etc. is widespread and increasing throughout the world. Many brain disorders have no observable structural signature and are not usually detected using biomedical sensors, but instead rely on subjective evaluation by trained clinicians. Until now, there has been no objective and quantitative clinical protocol or procedure which may be used for reliable medical diagnosis of these neurobehavioral disorders. An electroencephalograph (EEG) instrument (e.g., a medical sensor which non-invasively measures patterns of brain electrical activity) is a biomedical sensor for monitoring electrical signals and biomarkers from the body for clinical diagnosis of certain disorders such as epilepsy. Further exploitation of EEG biomarker processing may result in improved diagnosis of other neurobehavioral disorders and provide an objective means for better medical diagnosis.

### SUMMARY

The following presents a simplified summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In one aspect, the disclosure provides objective diagnosis of neurobehavioral disorders. Accordingly, the present disclosure discloses a method including: performing a spectral analysis on a Laplacian formatted electroencephalograph (EEG) data to generate a spectral coherence data; performing a plurality of regression analysis on the spectral coherence data to generate a smoothed spectral coherence data; performing a principal component analysis (PCA) on the smoothed spectral coherence data to generate an orthogonalized spectral coherence data; and performing a multivariate discriminant function analysis (DFA) on the orthogonalized spectral coherence data to generate a plurality of diagnostic rules and a diagnosis.

In one example, the method further includes computing the spectral coherence data using a van Drongelen technique. In one example, the method further includes generating a covariate data, wherein the covariate data describes a muscle artifact using the spectral coherence data from an electrode near a muscle. In one example, the method further includes generating a covariate data, wherein the covariate data describes an eye blink artifact using the spectral coherence data from an electrode near an eye.

In one example, the muscle artifact is described using a beta band in a range of 28-32 Hz from one or more electrode positions. In one example, the eye blink artifact is described using a slow delta band in a range of 0.5-1.0 Hz from one or more electrode positions. In one example, the orthogonalized spectral coherence data reduces a dimensionality of the smoothed spectral coherence data.

In one example, the method further includes performing a jackknifing procedure on the orthogonalized spectral coherence data to generate a cross-validated result for the plurality of diagnostic rules and the diagnosis. In one example, the method further includes performing a multiple split half replication on the orthogonalized spectral coherence data to evaluate classification success of the plurality of diagnostic rules and the diagnosis. In one example, the method further includes format-converting a refined electroencephalograph (EEG) data to generate the Laplacian formatted electroencephalograph (EEG) data.

In one example, the Laplacian formatted electroencephalograph (EEG) data is a scalp current potential data. In one example, the refined EEG data is generated using source analysis.

In one example, the method further includes removing a non-muscle-induced artifact from a filtered electroencephalograph (EEG) data to generate the refined electroencephalograph (EEG) data. In one example, the method further includes removing a muscle-induced artifact from an initially processed electroencephalograph (EEG) data to generate the filtered electroencephalograph (EEG) data. In one example, the method further includes removing a gross artifact in a raw electroencephalograph (EEG) data to generate the initially processed electroencephalograph (EEG) data. In one example, the method further includes collecting the raw electroencephalograph (EEG) data from a subject population.

In another aspect, the disclosure provides an apparatus including: means for performing a spectral analysis on a Laplacian formatted electroencephalograph (EEG) data to generate a spectral coherence data; means for performing a plurality of regression analysis on the spectral coherence data to generate a smoothed spectral coherence data; means for performing a principal component analysis (PCA) on the smoothed spectral coherence data to generate an orthogonalized spectral coherence data; and means for performing a multivariate discriminant function analysis (DFA) on the orthogonalized spectral coherence data to generate a plurality of diagnostic rules and a diagnosis.

In one example, the apparatus further includes: means for performing a jackknifing procedure on the orthogonalized spectral coherence data to generate a cross-validated result for the plurality of diagnostic rules and the diagnosis; and means for performing a multiple split half replication on the orthogonalized spectral coherence data to evaluate classification success of the plurality of diagnostic rules and the diagnosis.

In one example, the apparatus further includes: means for format-converting a refined electroencephalograph (EEG) data to generate the Laplacian formatted electroencephalograph (EEG) data; means for removing a non-muscle-induced artifact from a filtered electroencephalograph (EEG) data to generate the refined electroencephalograph (EEG) data; and means for removing a muscle-induced artifact from an initially processed electroencephalograph (EEG) data to generate the filtered electroencephalograph (EEG) data.

In one example, the apparatus further includes: means for removing a gross artifact in a raw electroencephalograph (EEG) data to generate the initially processed electroencephalograph (EEG) data; and means for collecting the raw electroencephalograph (EEG) data from a subject population.

These and other aspects of the present disclosure will become more fully understood upon a review of the detailed description, which follows. Other aspects, features, and implementations of the present disclosure will become apparent to those of ordinary skill in the art, upon reviewing the following description of specific, exemplary implementations of the present invention in conjunction with the accompanying figures. While features of the present invention may be discussed relative to certain implementations and figures below, all implementations of the present invention can include one or more of the advantageous features discussed herein. In other words, while one or more implementations may be discussed as having certain advantageous features, one or more of such features may also be used in accordance with the various implementations of the invention discussed herein. In similar fashion, while exemplary implementations may be discussed below as device, system, or method implementations it should be understood that such exemplary implementations can be implemented in various devices, systems, and methods.

### BRIEF DESCRIPTION OF THE DRAWINGS

FIG. 1 illustrates an example electroencephalograph (EEG) analysis flow process.

FIG. 2 illustrates an example of standard electroencephalograph (EEG) electrode names and positions.

FIG. 3 illustrates an example of raw electroencephalograph (EEG) data which contains visually obvious eye-blink and muscle artifacts.

FIG. 4 illustrates an example of filtered electroencephalograph (EEG) data where muscle-induced artifacts have been removed using low pass spectral filtering.

FIG. 5 illustrates an example of refined electroencephalograph (EEG) data after source analysis with eye-blink artifacts shown.

FIG. 6 illustrates an example of refined electroencephalograph (EEG) data after source analysis with eye artifacts removed.

FIG. 7 illustrates an example of performing principal component analysis on the smoothed spectral coherence data.

FIG. 8 illustrates an example of discriminant function analysis (DFA).

FIG. 9 illustrates an example flow diagram for objective diagnosis of neurobehavioral disorders.

FIG. 10 illustrates an example information processing system.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

While for purposes of simplicity of explanation, the methodologies are shown and described as a series of acts, it is to be understood and appreciated that the methodologies are not limited by the order of acts, as some acts may, in accordance with one or more aspects, occur in different orders and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with one or more aspects.

Brain dysfunction is widespread and affects numerous families in the United States and globally. (For example, more than 1 billion of the world's population suffer from neurological disorders and nearly 1 billion suffer from mental disorders. Consequently, 1 in 4 people worldwide will be affected by neurological or mental disorders in their lifetime.) Common disorders include Autism Spectrum Disorder (ASD)/Asperger's Syndrome, Attention Deficit Disorder (ADD), Dyslexia, Dementia, Alzheimer's Disease, Chronic Fatigue Syndrome, Schizophrenia, and many others. Since many brain disorders have no observable structural anomalies, they have not been detectable using CT (Computed Tomography) or MRI (Magnetic Resonance Imaging) equipment, whose primary value to date has been in detecting anatomical or structural abnormalities.

EEG (Electroencephalography) monitors electrical signals from the brain and is primarily used to diagnose epilepsy and tumors. EEG typically relies on a clinician's visual examination of EEG data to make these diagnoses. The human eye, however, is not able to analyze the complexity of the aforementioned brain disorders' data signatures and patterns. Instead, diagnoses of the above list of disorders relies on subjective evaluation by trained clinicians. Until now, there has been no objective and quantitative clinical protocol which allows reliable medical diagnosis of these neurobehavioral disorders. Objective, biomarker-based diagnostic tests are virtually non-existent in the neurobehavior field, resulting in inaccurate diagnoses that can lead to misdirected treatments, heartache, hardship, and great financial cost for families.

However, EEG data, if analyzed using a specific mathematical or statistical protocol, may identify disease-specific biomarkers in neurobehavioral disorders that have previously been undetectable by the medical community. This methodology first analyzes and filters raw EEG data, removing the extraneous signals and artifacts that can obfuscate important underlying EEG data, and then utilizes statistical and mathematical analysis to identify these disorder-specific biomarkers.

In one example, EEG signals as a function of time (i.e., temporal EEG signals) acquired from a plurality of spatial locations using electrode sensors are perceptive biomedical signals. For example, EEG signals may be used as biomarkers, that is, measurements which provide an accurate monitoring of various biological functions and processes. EEG signals may be processed to extract biomedical information from the body and be used as biomarkers. However, collecting, processing and analyzing EEG signals from the brain may be challenging for a variety of reasons. First, acquired EEG signals may be contaminated by external interfering signals that do not arise directly from brain activity but from external movements such as eye blink and muscle movement. Such external interfering signals may serve as false indicators of neurological activity and therefore must be carefully minimized or removed from the acquired EEG signals. Until recently, it has been difficult to eliminate such external interfering signals to allow improved analysis of the underlying desired EEG data in the acquired EEG signals. The present invention is oriented towards using direct visualization and signal analysis, spectral filtering, source analysis and regression analysis to result in improved EEG biomarker processing and therefore a more objective diagnosis of neurobehavioral disorders.

In addition, analysis of acquired EEG signals relies on carefully controlled data collection protocols and requires data specialists with expertise in a number of diverse fields such as engineering, mathematics, statistics and software development to properly interpret the EEG data. One goal for successful EEG data analysis is to reveal and identify functional abnormalities beyond detection of structural abnormalities. In particular, successful EEG data analysis should incorporate automatic signal analysis to replace manual marking and removal processes to eliminate contamination from external interfering signals. EEG data analysis should also include machine learning and artificial intelligence (AI) for refinement and enhancement of EEG signal processing.

In one example, analysis of acquired EEG signals may incorporate statistical processing to minimize extraneous influence of external interfering signals due to, for example, eye blinking, electrode shorting, muscle movement, etc. The statistical processing analysis may also include spectral coherence measurements and analysis of spatially diverse portions of the brain for brain pattern recognition using spectral coherence patterns. Such spectral coherence patterns may be classified as being normal or abnormal and may be calibrated against neurotypical controls to provide a neurological diagnostic scoring. In addition, updated EEG signals may be incorporated over time to refine and enhance the diagnostic scoring via machine learning.

In one example, an EEG analysis process includes the following steps:
1. Extraction and replication of a priori diagnostic and statistical rules to generate mathematical rules for individual diagnosis.
2. Signal analysis to replace visual inspection of acquired EEG data to eliminate interference and artifacts.
3. Augmentation of existing EEG data with updated EEG data as it become available.
4. Definition of classification rules for additional brain disorders.
   One skilled in the art would understand that one or more of the listed steps may be deleted or that the order of the steps may be altered and still be within the spirit and scope of the present disclosure. One skilled in the art would understand that the listed steps are not exclusive and that other step(s) may be added within the spirit and scope of the present disclosure.

In one example, the EEG analysis process may diagnose the following disorders:
1. Autistic spectrum disorder (ASD), Aspergers syndrome
2. Attention deficit disorder
3. Chronic fatigue syndrome
4. Alzheimer's disease
5. Schizophrenia prodrome
6. Dementia
7. Chronic traumatic encephalopathy (CTE)
8. Post traumatic stress disorder (PTSD)
9. Bipolar disorder
10. Anxiety disorder
11. Depression
12. Sociopathy
13. Long term Covid
14. Dyslexia
   One skilled in the art would understand that the listed disorders are not exclusive and that other disorders may be added within the spirit and scope of the present disclosure.

In one example, the EEG analysis process collects raw EEG data from carefully screened human subjects and processes the raw EEG data through a series of signal processing functions which result in spectral coherence data. The spectral coherence data may next be processed through a series of statistical functions which results in a plurality of disorder-specific biomarker data.

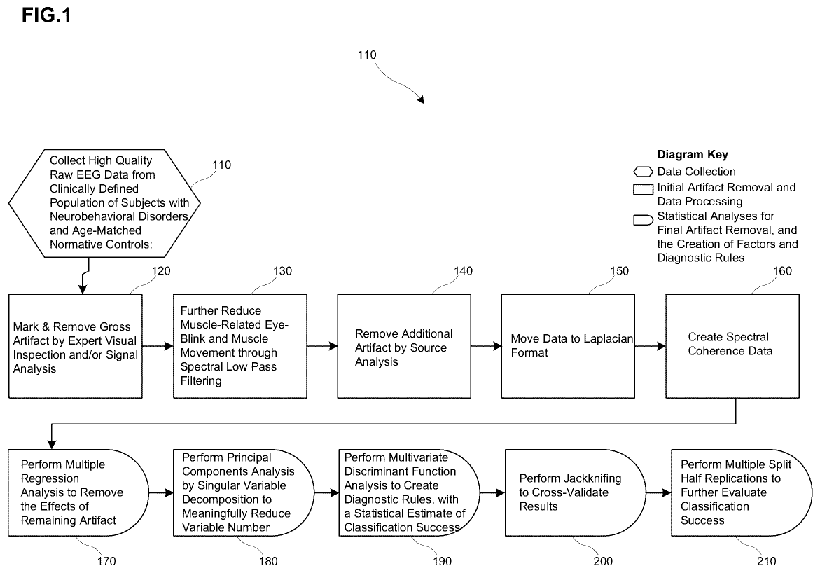

FIG. 1 illustrates an example electroencephalograph (EEG) analysis flow process 100. The process starts in block 110 by collecting raw EEG data from a clinically defined subject population. In one example, the clinically defined subject population includes individuals with neurobehavioral disorders and age-matched normative controls.

For example, a disordered group includes only patients who have experienced a disorder and where expert clinicians have recognized and identified patients with a disorder, incorporating criteria defined in DSM-IV (Diagnostic and Statistical Manual of Mental Disorders) and/or ADOS (autism diagnostic observation schedule). For example, exclusion from a disorder group may include patients with co-existing primary neurological syndromes which present as disease features, clinical seizure disorders or EEG reading results which suggest an active seizure disorder or epileptic encephalopathy. In one example, exclusion from a disorder group may also include a primary diagnosis of global developmental delay (GDD), expressed doubt by the referring clinicians regarding the diagnosis, medicine consumption at time of the EEG study, significant primary sensory disorders such as blindness, deafness, etc.

For example, a normative population may be age-matched and selected to be normally functioning, while avoiding the creation of an exclusively super-normal group (e.g., low birth weight children not needing subsequent medical attention are included in the normal population). For example, exclusion from a normative population may be individuals with diagnosed or suspected neurological or psychiatric illness or disorder, abnormal neurological examination as identified during research study, clinical seizure disorders or EEG reading results which suggest an active seizure disorder or epileptic encephalopathy, disordered features, newborn period diagnosis of intraventricular hemorrhage (IVH), retinopathy of prematurity, hydrocephalus, cerebral palsy or other significant condition which influences EEG data, medicine consumption a time of the EEG study, etc.

For example, the raw EEG data may utilize a plurality of EEG channels (e.g., 24 channels) on EEG equipment with specific sensor placement by trained technicians. In one example, the trained technicians may work with the patient to mitigate extraneous artifacts in the raw EEG data.

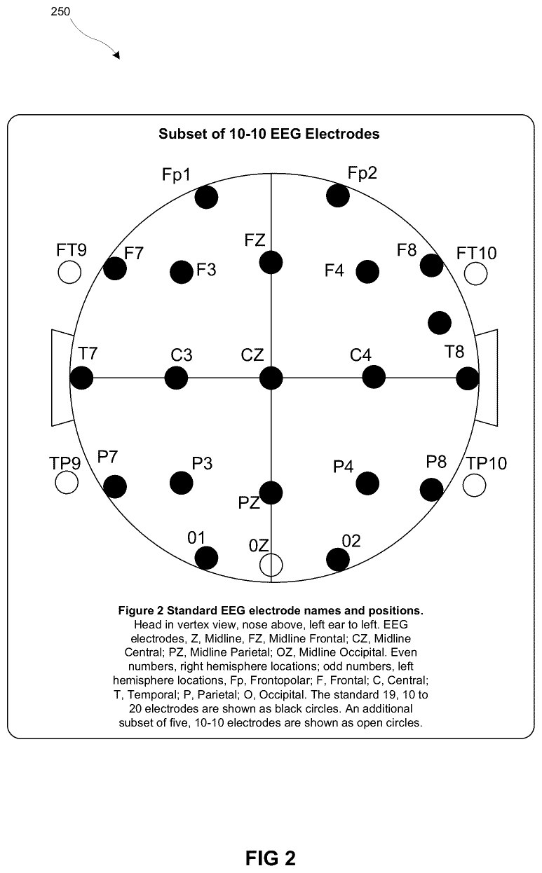

FIG. 2 illustrates an example 250 of standard electroencephalograph (EEG) electrode names and positions. In one example, the raw EEG data may be collected using electrodes placed according to a standard methodology (e.g., 10-20 EEG electrode placement position methodology), as illustrated in FIG. 2.

The process continues in block 120 by marking and removing gross artifacts from the raw EEG data to generate initially processed EEG data. In one example, the marking and removal of gross artifacts employs expert visual inspection and/or signal analysis.

In one example, gross artifact removal employs artifact signal templates where signal matches may be filtered out of the raw EEG data. In one example, gross artifacts include eye blink storms, position/movement adjustment, time outs, upsets due to tensing, stretching, yawning, momentary interrupts, abnormal states (e.g., drowsiness or sleep), electrode artifacts (e.g., electrical shorts, bad electrodes) and normal EEG transients, etc.

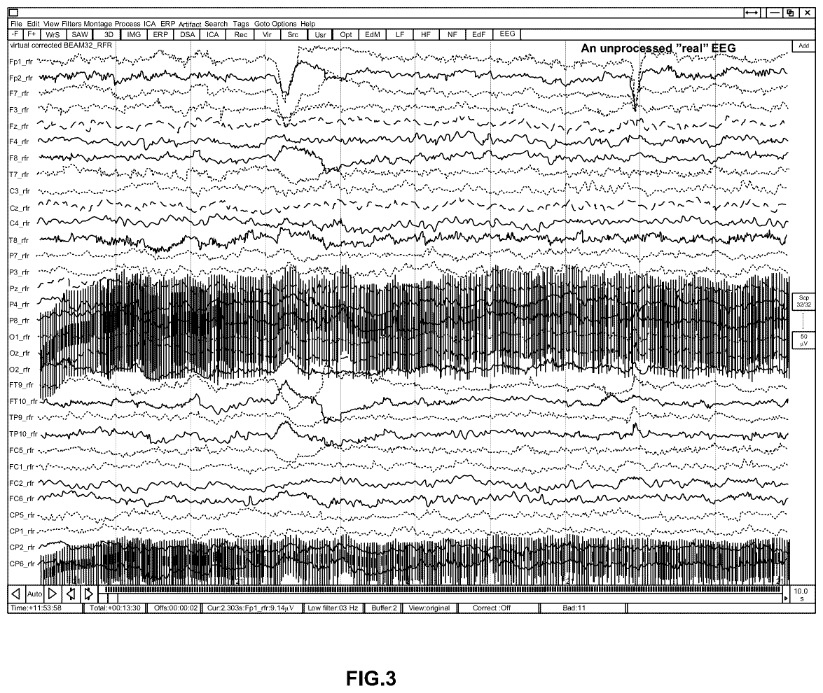

FIG. 3 illustrates an example 300 of raw electroencephalograph (EEG) data which contains visually obvious eye-blink and muscle artifacts. The process continues in block 130 by removing muscle-induced artifacts from the initially processed EEG data to generate filtered EEG data. In one example, the muscle-induced artifacts include muscle-related eye blink and muscle movement.

In one example, muscle-induced artifacts may be removed using low pass spectral filtering (e.g., using a 40 Hz low pass spectral filter). In one example, power line interference may also be removed using notch filtering (e.g., using a 60 Hz notch filter).

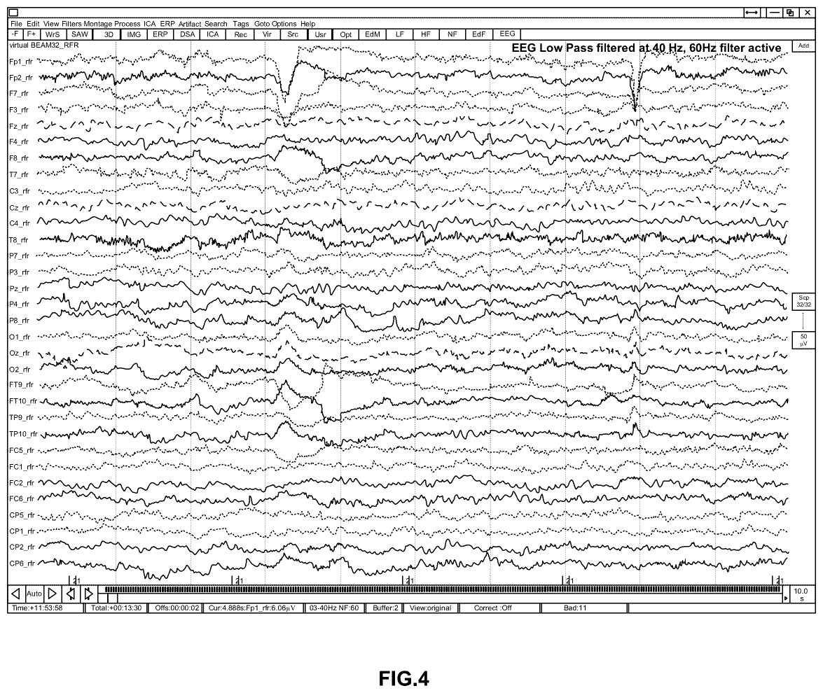

FIG. 4 illustrates an example 400 of filtered electroencephalograph (EEG) data where muscle-induced artifacts have been removed using low pass spectral filtering. The process continues in block 140 by removing non-muscle-induced artifacts from the filtered EEG data to generate refined EEG data. In one example, the refined EEG data are generated using source analysis. In one example, the non-muscle-induced artifacts include eye-blink artifacts. In one example, the non-muscle-induced artifacts may be removed using a Berg-Scherg technique as part of source analysis. For example, the Berg-Scherg technique employs a multiple source eye correction procedure which models spatial distribution of eye activity using calibration data. In one example, the refined EEG data may include frontal low amplitude delta signals.

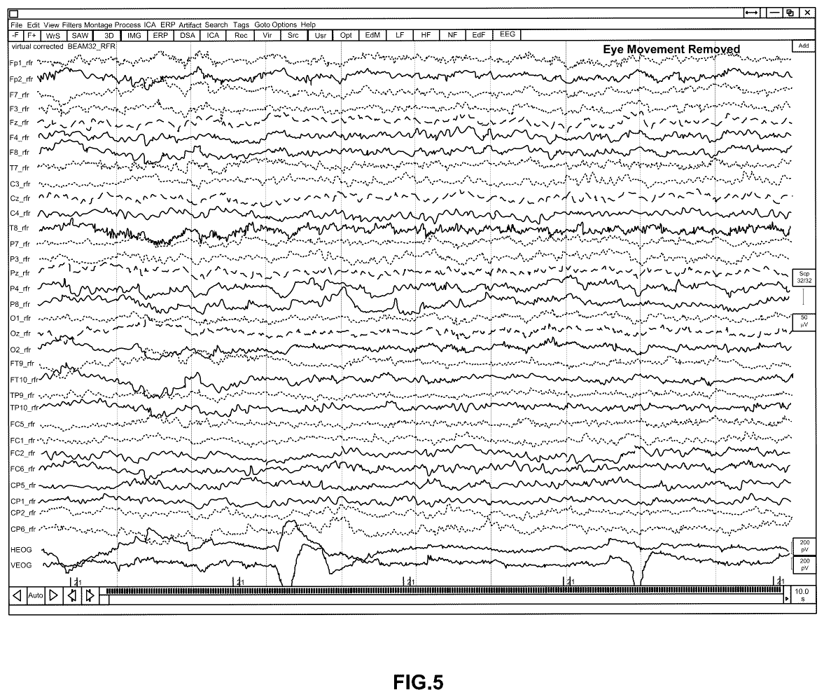

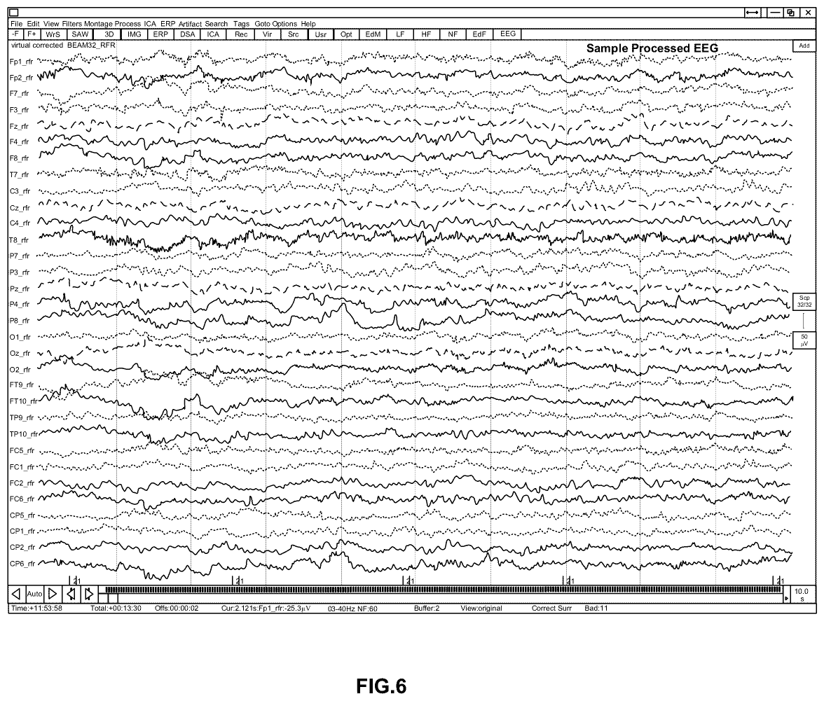

FIG. 5 illustrates an example 500 of refined electroencephalograph (EEG) data after source analysis with eye-blink artifacts shown. FIG. 6 illustrates an example 600 of refined electroencephalograph (EEG) data after source analysis with eye artifacts removed.

The process continues in block 150 by converting the refined EEG data into a Laplacian format to generate formatted EEG data. In one example, the Laplacian format transforms the refined EEG data into scalp current potential data. In one example, the formatted EEG data may include a plurality of specific channels each containing a plurality of spectral bands using three-dimensional spatial interpolation.

The process continues in block 160 by performing spectral analysis on the formatted EEG data to generate spectral coherence data. In one example, the spectral coherence data is computed using a van Drongelen technique. For example, spectral coherence data is defined as a cross-spectrum normalized by a square root of a product of two auto-spectra. For example, spectral coherence data is a complex-valued quantity. For example, a coherence metric may be defined as a square modulus of the spectral coherence data, with values ranging between zero and unity. For example, a coherence metric of unity indicates highly coherent electrodes and a coherence metric of zero indicates electrodes with no coherence. For example, spectral coherence data may be indexed over spectral coherence variables. For example, spectral coherence variables may be spatial variables and spectral variables. In one example, the cross-spectrum is a Fourier transform of a cross-correlation function between two spatially separated EEG signals. In one example, the auto-spectrum is a Fourier transform of an auto-correlation function of an individual EEG signal.

In one example, spectral coherence data may be generated over a specified frequency range with a quantity of spectral bands having a spectral resolution. For example, the specified frequency range may be 1-32 Hz and the quantity of spectral bands may have a spectral resolution of 2 Hz.

For example, with 24 electrode positions and a quantity of 16 spectral bands, there would be a total of 4416 unique spectral coherence variables. That is, with 24 electrode positions, there are a total of 24×24=576 possible coherence spatial pairs, including 24 self-coherence spatial pairs, which results in 552 remaining coherence spatial pairs. Of the 552 remaining coherence spatial pairs, half are redundant with the other half, resulting in 276 unique coherence spatial pairs per spectral band. That is, with 16 spectral bands, there are a total of 4416 unique spectral coherence variables.

In general, for N electrode positions and M spectral bands, the total number Q of unique spectral coherence variables is given by Q=½ N(N−1)M. For example, the total number Q of unique spectral coherence variables increases as N² asymptotically.

The process continues in block 170 by performing multiple regression analysis on the spectral coherence data to generate smoothed spectral coherence data. In one example, the multiple regression analysis creates covariate data which describes remaining muscle and eye blink artifacts using spectral coherence data from electrodes near muscles and eyes. For example, muscle artifacts may be described with 4 variables using beta band, 28-32 Hz, from electrode positions FP1+FP2, F7+F8, T7-T8, P7-P8. For example, eye artifacts may be described with 2 variables using slow delta band, 0.5-1.0 Hz, from electrode positions FP1+FP2, F7+F8. For example, the smoothed spectral coherence data may be generated using common average data with six covariate or nuisance variables created.

For example, 6 nuisance variables may be treated as independent variables and 4416 coherence variables may be treated as dependent variables in the multiple regression analysis. For example, the 4416 coherence variables or residuals may be used for subsequent analysis since they will have nearly zero correlation with the 6 nuisance variables (i.e., clear of eye blink slow delta band data and muscle beta data).

The process continues in block 180 by performing principal component analysis (PCA) on the smoothed spectral coherence data to generate orthogonalized spectral coherence data. In one example, the orthogonalized spectral coherence data reduce a dimensionality of the smoothed spectral coherence data (e.g., to less than 100 important factors) comprised of correlated variables. In one example, the orthogonalized spectral coherence data may be used to create factor data which then generate rules (not subject to investigator bias) and dependent only on number and character of utilized subjects. For example, the number of factors may be as high as number of correlated variables, and the variance (e.g., information content) is biased by the first factor and less affected by the last factor.

For example, data reduction may be performed by choosing a subset of the factor data (e.g., top factor set) which explains a desired total content in the aggregate. For example, each factor represents a weighted sum of a selected set of input variables. For example, factor loadings may be used to decode factor meaning.

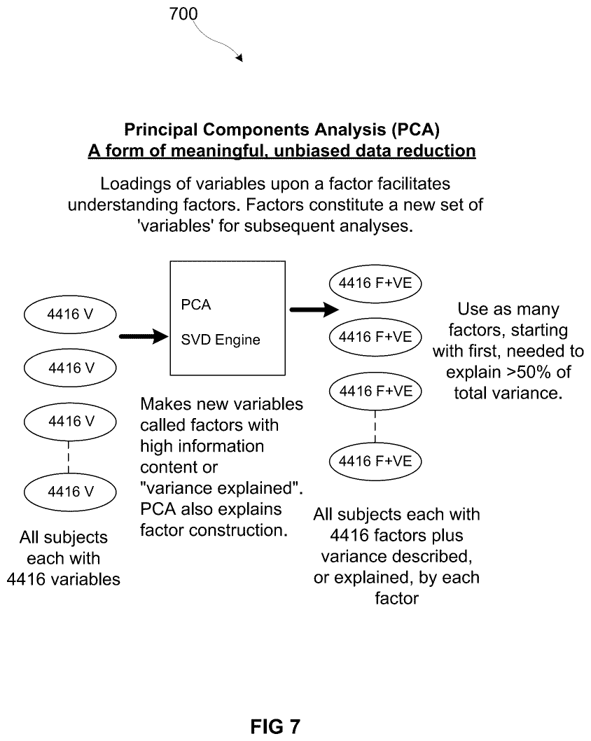

In one example, FIG. 7 illustrates an example 700 of performing principal component analysis on the smoothed spectral coherence data. The process continues in block 190 by performing multivariate discriminant function analysis (DFA) on the orthogonalized spectral coherence data to generate diagnostic rules. In one example, the diagnostic rules include a statistical estimate of classification success. For example, the orthogonalized spectral coherence data may be characterized and compared to a control and disordered group and then mapped against clusters of normal group and abnormal group.

For example, each subject of the clinically defined subject population may be placed onto a continuum vs. normal reference. For example, group membership may be predicted from a set of input predicator variables. For example, a discriminant function may be created to generate an optimal linear combination of input discriminating variables. For example, significance of group discrimination may be determined and indication of success of a subject to group classification may be provided.

In one example, input variables may be selected by the DFA algorithm (stepping) which permits identification of most valuable input variables and may also produce best discriminant functions. For example, a prospective test of group classification robustness follows creating multiple random training and test sets and evaluating classification success for each set pair. For example, new test subjects may be injected into the group to classify while following the same process as all previous test subjects such that rules are not affected.

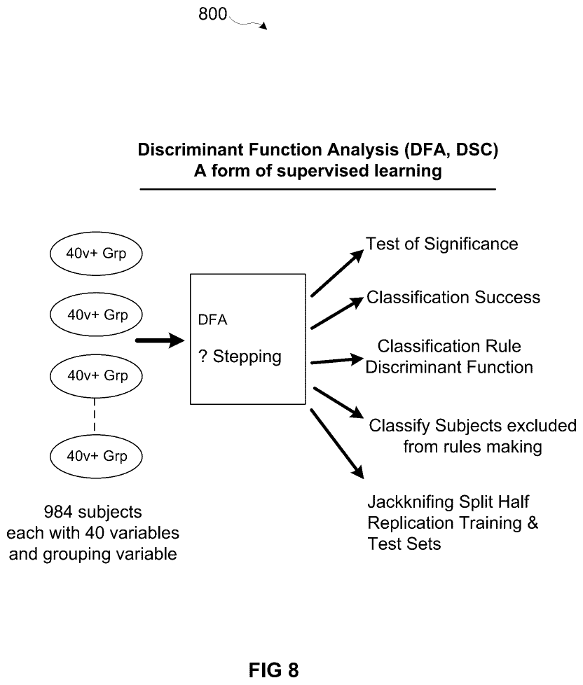

In one example, FIG. 8 illustrates an example 800 of discriminant function analysis (DFA). For example, DFA is one form of supervised learning. The process continues in block 200 by performing jackknifing processing on the orthogonalized spectral coherence data to generate cross-validated results. In one example, jackknifing processing involves rerun of statistical tests with one individual component removed which is then classified. For example, the rerun is repeated with each other individual component removed.

The process continues in block 210 by performing multiple split half replication on the orthogonalized spectral coherence data to evaluate classification success. In one example, all individuals of the original study are randomly partitioned into a plurality of groups. For example, the plurality of groups is selected where one half of the normal group becomes a rule-making group and the other half of the normal group becomes a test group. For example, the plurality of groups is selected where one half of the abnormal group becomes the rule-making group and the other half of the abnormal group becomes the test group. For example, the statistical tests are run on the plurality of groups to determine if the established rules are still applicable. For example, the split half replication may be repeated a number of times (e.g., 10 times) with group membership selected by a random number generator.

EEG biomarker processing may be applied to many neurobehavioral disorders to obtain objective diagnostic results based on raw EEG data after processing. For example, disease biomarkers can be identified before full symptoms occur to allow more timely and effective treatment. For example, diagnostic results may be presented on a continuum scale which shows deviation from a normative baseline scale to allow for more accurate targeted treatments. Results may be used to measure treatment intervention efficacy and may also identify affected brain areas. Biomarker identification may be used for additional disorders as well.

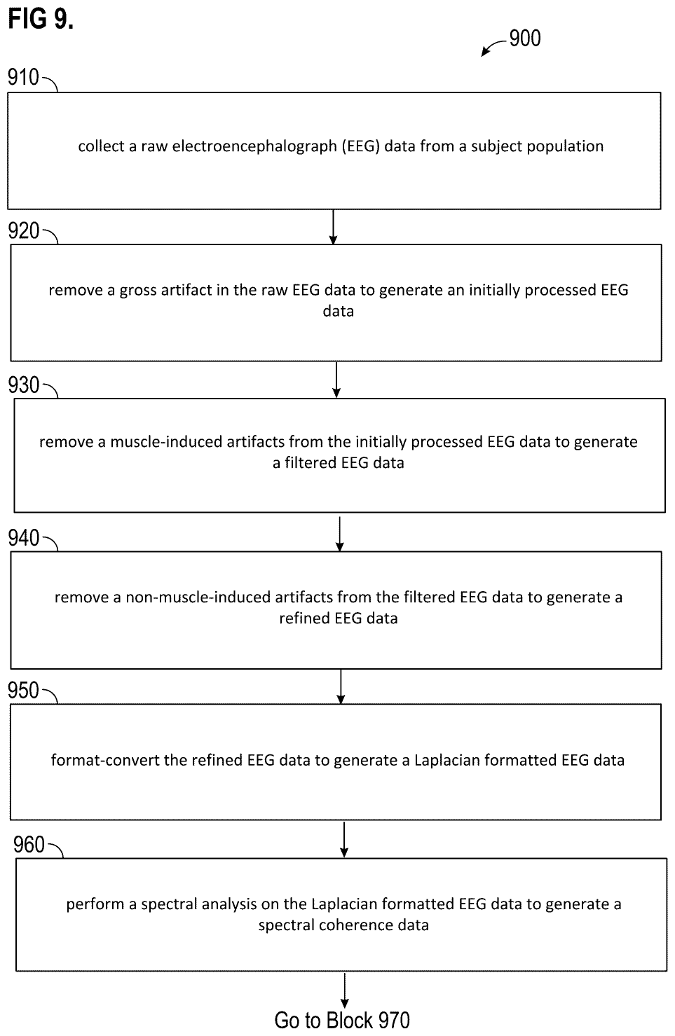

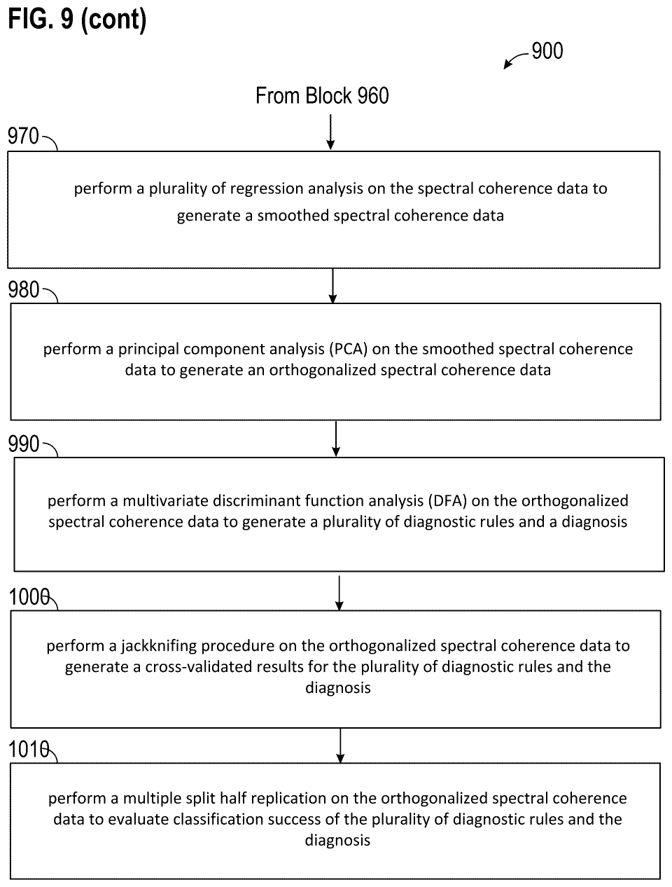

FIG. 9 illustrates an example flow diagram 900 for objective diagnosis of neurobehavioral disorders. In block 910, collect a raw electroencephalograph (EEG) data from a subject population. In one example, the subject population is a clinically defined subject population. In one example, the clinically defined subject population includes individuals with neurobehavioral disorders and age-matched normative controls. In one example, the raw EEG data may utilize a plurality of EEG channels (e.g., 24 channels) on EEG equipment. In one example, the raw EEG data may be collected using electrodes placed according to a 10-20 EEG electrode placement position methodology). In one example, the raw EEG data is collected by an electroencephalograph (EEG) instrument.

In block 920, remove a gross artifact in the raw EEG data to generate an initially processed EEG data. In one example, the gross artifact is marked in the raw EE data prior to its removal. In one example, removal of gross artifacts employs artifact signal templates to allow signal matching and filtering of the raw EEG data. In one example, gross artifacts include eye blink storms, position and movement adjustment, time outs, upsets due to tensing, stretching, yawning, momentary interrupts, abnormal states (e.g., drowsiness or sleep), electrode artifacts (e.g., shorts, bad electrodes), normal EEG transients, etc. In one example, the generation of the initially processed EEG data may be performed by a digital signal processing (DSP) engine.

In block 930, remove a muscle-induced artifact from the initially processed EEG data to generate a filtered EEG data. In one example, the muscle-induced artifacts include muscle-related eye blink and muscle movement. In one example, the removal of muscle-induced artifacts uses low pass spectral filtering to remove muscle-induced artifacts. In one example, the low pass spectral filtering may use a 40 Hz low pass spectral filter. In one example, the removal of muscle-induced artifacts may use notch filtering (e.g., using a 60 Hz notch filter to remove power line interference). In one example, the generation of the filtered EEG data may be performed by a digital signal processing (DSP) engine.

In block 940, remove a non-muscle-induced artifacts from the filtered EEG data to generate a refined EEG data. In one example, the refined EEG data are generated using source analysis. In one example, source analysis determines a spatial distribution of artifact sources. In one example, the non-muscle-induced artifacts include eye-blink artifacts. In one example, the non-muscle-induced artifacts may be removed using a Berg-Scherg technique as part of source analysis. For example, the Berg-Scherg technique employs a multiple source eye correction procedure which models spatial distribution of eye activity using calibration data. In one example, the refined EEG data may include frontal low amplitude delta signals. In one example, the generation of the refined EEG data may be performed by a digital signal processing (DSP) engine.

In one example, frontal low amplitude delta signals are signals due to low amplitude vertical and horizontal eye movements in the delta frequency band. In one example, frontal low amplitude delta signals follow eyeball motion in response to unconscious brain stimulus.

In block 950, format-convert the refined EEG data to generate a Laplacian formatted EEG data. In one example, the Laplacian format transforms the refined EEG data into a scalp current potential data. That is, in one example, the Laplacian formatted EEG data is a scalp current potential data. In one example, the formatted EEG data may include a plurality of specific channels each containing a plurality of spectral bands using three-dimensional spatial interpolation. In one example, the scalp current potential data relies on an ohmic relationship between the scalp current potential data and the refined EEG data. For example, the ohmic relationship is a linear relation between current and voltage. In one example, the generation of the Laplacian formatted EEG data may be performed by a digital signal processing (DSP) engine.

In block 960, perform a spectral analysis on the Laplacian formatted EEG data to generate a spectral coherence data. In one example, the spectral coherence data is computed using a van Drongelen technique. For example, spectral coherence data is defined as a cross-spectrum normalized by a square root of a product of two auto-spectra. For example, spectral coherence data is a complex-valued quantity. For example, a coherence metric may be defined as a square modulus of the spectral coherence data, with values ranging between zero and unity. For example, a coherence metric of unity indicates highly coherent electrodes and a coherence metric of zero indicates electrodes with no coherence. For example, spectral coherence data may be indexed over spectral coherence variables. For example, spectral coherence variables may be spatial variables and spectral variables. In one example, the generation of the spectral coherence data may be performed by a digital signal processing (DSP) engine.

In one example, the van Drongelen technique may be used to determine brain source locations of EEG activity recorded at the scalp level. In one example, it may be part of a source analysis algorithm for EEG signal analysis.

In one example, spectral coherence data may be generated over a specified frequency range with a quantity of spectral bands having a spectral resolution. For example, the specified frequency range may be 1 to 32 Hz and the quantity of spectral bands may have a spectral resolution of 2 Hz.

In block 970, perform a plurality of regression analysis on the spectral coherence data to generate a smoothed spectral coherence data. In one example, the plurality of regression analysis generates covariate data describing muscle and eye blink artifacts using spectral coherence data from electrodes near muscles and eyes. For example, muscle artifacts may be described with four variables using a beta band, 28-32 Hz, from 4 electrode positions. In one example, the quantity of variables is related to the quantity of electrode positions.

For example, eye blink artifacts may be described with two variables using a slow delta band, 0.5-1.0 Hz, from 2 electrode positions. In one example, the quantity of variables is related to the quantity of electrode positions. For example, the smoothed spectral coherence data may be generated using common average data with six covariate or nuisance variables created. For example, the 6 nuisance variables may be treated as independent variables and other coherence variables may be treated as dependent variables in the multiple regression analysis. In one example, the generation of the smoothed spectral coherence data may be performed by a digital signal processing (DSP) engine.

In block 980, perform a principal component analysis (PCA) on the smoothed spectral coherence data to generate an orthogonalized spectral coherence data. In one example, the orthogonalized spectral coherence data reduce a dimensionality of the smoothed spectral coherence data comprised of correlated variables. In one example, the orthogonalized spectral coherence data may be used to create a plurality of factors for rules generation. For example, a number of factors of the plurality of factors may be equal to a number of correlated variables. For example, a variance (e.g., information content) of the orthogonalized spectral coherence data may be biased by a first factor of the plurality of factors and less affected by a last factor of the plurality of factors. For example, data reduction may be performed by choosing a subset of the plurality of factors. In one example, the generation of the orthogonalized spectral coherence data may be performed by a digital signal processing (DSP) engine.

In block 990, perform a multivariate discriminant function analysis (DFA) on the orthogonalized spectral coherence data to generate a plurality of diagnostic rules and a diagnosis. In one example, the diagnostic rules include a statistical estimate of classification success; that is, for example, the success rate of a particular diagnosis. For example, the orthogonalized spectral coherence data may be characterized and compared to a control and disordered group and then mapped against clusters of a normal group of subjects and an abnormal group of subjects. In one example, the generation of the plurality of diagnostic rules may be performed by a central processing unit (CPU).

In one example, Discriminant Function Analysis (DFA) is performed on a group of already diagnosed normal and abnormal subjects to produce a training set used generate a plurality of diagnostic rules. In one example, the plurality of diagnostic rules may then be applied for diagnosis of a diagnosed individual subject.

For example, a subject of the normal group of subjects and a subject of the abnormal group of subjects may be placed onto a continuum vs. normal reference. For example, group membership may be predicted from a set of input predicator variables. For example, a discriminant function may be created to generate an optimal linear combination of input variables. In one example, input variables may be selected by the DFA which permits identification of most valuable input variables and may also produce optimal discriminant functions. In one example, the diagnosis may include one of the following disorders: autistic spectrum disorder (ASD), Aspergers syndrome, attention deficit disorder, chronic fatigue syndrome, Alzheimer's disease, schizophrenia prodrome, dementia, chronic traumatic encephalopathy (CTE), post-traumatic stress disorder (PTSD), bipolar disorder, anxiety disorder, depression, sociopathy, long term Covid, dyslexia, etc.

In block 1000, perform a jackknifing procedure on the orthogonalized spectral coherence data to generate a cross-validated result for the plurality of diagnostic rules and the diagnosis. In one example, the orthogonalized spectral coherence data includes a plurality of spectral components. In one example, the jackknifing processing executes statistical tests on the orthogonalized spectral coherence data with one spectral component of the plurality of spectral components removed. For example, the execution of statistical tests is repeated with each spectral component of the plurality of spectral components removed. In one example, the generation of the cross-validated result may be performed by a central processing unit (CPU).

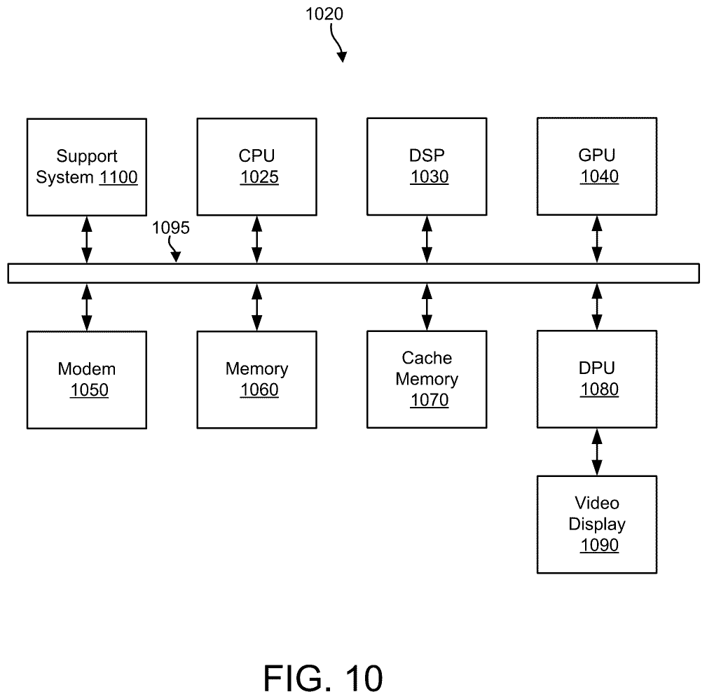

In block 1010, perform a multiple split half replication on the orthogonalized spectral coherence data to evaluate classification success of the plurality of diagnostic rules and the diagnosis. In one example, each individual of the subject population is randomly partitioned into a group of a plurality of groups. For example, the plurality of groups is selected where a first half of a normal group is a rule-making group and a second half of the normal group is a test group. For example, the plurality of groups is selected where a first half of the abnormal group becomes the rule-making group and a second half of the abnormal group becomes the test group. For example, the statistical tests are run on the plurality of groups to determine if the plurality of diagnostic rules are applicable. For example, the split half replication may be repeated a number of times (e.g., 10 times) with group membership selected by a random number generator. In one example, the evaluation of classification success may be performed by a central processing unit (CPU). In one example, the steps illustrated in FIG. 9 may be performed by one or more components illustrated in FIG. 10.

FIG. 10 illustrates an example information processing system 1020. In one example, the information processing system 1020 includes a plurality of processing engines such as a central processing unit (CPU) 1025, a digital signal processor (DSP) 1030, a graphics processing unit (GPU) 1040, a display processing unit (DPU) 1080, etc. In one example, various other functions in the information processing system 1000 may be included such as a support system 1100, a modem 1050, a memory 1060, a cache memory 1070 and a video display 1090. For example, the plurality of processing engines and various other functions may be interconnected by an interconnection databus 1095 to transport data and control information.

In one example, the CPU 1020 may be used for generic processing tasks without intensive numerical computations or without substantial parallelism. In one example, the DSP 1030 may be used for intensive numerical computations such as signal processing operations, including Fourier transformation, spectral analysis, coherence spectrum generation, statistical calculations, etc. In one example, the GPU 1040 may be used for highly parallel calculations such as performed for three-dimensional (3D) graphical applications. In one example, the DPU 1050 may be used for rendering two-dimensional images onto the video display 1090. Although several components of the information processing system 1020 are included herein, one skilled in the art would understand that the components listed herein are examples and are not exclusive. Thus, other components may be included as part of the information processing system 1000 within the spirit and scope of the present disclosure. In one example, the various components shown in FIG. 10 may be used to perform one or more steps illustrated in FIG. 9.

In one aspect, one or more of the steps for providing objective diagnosis of neurobehavioral disorders in FIG. 9 may be executed by one or more processors which may include hardware, software, firmware, etc. The one or more processors, for example, may be used to execute software or firmware needed to perform the steps in the flow diagram of FIG. 9. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

The software may reside on a computer-readable medium. The computer-readable medium may be a non-transitory computer-readable medium. A non-transitory computer-readable medium includes, by way of example, a magnetic storage device (e.g., hard disk, floppy disk, magnetic strip), an optical disk (e.g., a compact disc (CD) or a digital versatile disc (DVD)), a smart card, a flash memory device (e.g., a card, a stick, or a key drive), a random access memory (RAM), a read only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The computer-readable medium may also include, by way of example, a carrier wave, a transmission line, and any other suitable medium for transmitting software and/or instructions that may be accessed and read by a computer. The computer-readable medium may reside in a processing system, external to the processing system, or distributed across multiple entities including the processing system. The computer-readable medium may be embodied in a computer program product. By way of example, a computer program product may include a computer-readable medium in packaging materials. The computer-readable medium may include software or firmware. Those skilled in the art will recognize how best to implement the described functionality presented throughout this disclosure depending on the particular application and the overall design constraints imposed on the overall system.

Any circuitry included in the processor(s) is merely provided as an example, and other means for carrying out the described functions may be included within various aspects of the present disclosure, including but not limited to the instructions stored in the computer-readable medium, or any other suitable apparatus or means described herein, and utilizing, for example, the processes and/or algorithms described herein in relation to the example flow diagram.

Within the present disclosure, the word “exemplary” is used to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The term “coupled” is used herein to refer to the direct or indirect coupling between two objects. For example, if object A physically touches object B, and object B touches object C, then objects A and C may still be considered coupled to one another-even if they do not directly physically touch each other. The terms “circuit” and “circuitry” are used broadly, and intended to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in the figures may be rearranged and/or combined into a single component, step, feature or function or embodied in several components, steps, or functions. Additional elements, components, steps, and/or functions may also be added without departing from novel features disclosed herein. The apparatus, devices, and/or components illustrated in the figures may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

One skilled in the art would understand that various features of different embodiments may be combined or modified and still be within the spirit and scope of the present disclosure.

What is claimed is:

1. A method comprising:
performing a spectral analysis on a Laplacian formatted electroencephalograph (EEG) data to generate a spectral coherence data;
performing a plurality of regression analysis on the spectral coherence data to generate a smoothed spectral coherence data;
performing a principal component analysis (PCA) on the smoothed spectral coherence data to generate an orthogonalized spectral coherence data; and
performing a multivariate discriminant function analysis (DFA) on the orthogonalized spectral coherence data to generate a plurality of diagnostic rules and a diagnosis.

2. The method of claim 1, further comprising computing the spectral coherence data using a van Drongelen technique.

3. The method of claim 1, further comprising generating a covariate data, wherein the covariate data describes a muscle artifact using the spectral coherence data from an electrode near a muscle.

4. The method of claim 3, wherein the muscle artifact is described using a beta band in a range of 28-32 Hz from one or more electrode positions.

5. The method of claim 1, further comprising generating a covariate data, wherein the covariate data describes an eye blink artifact using the spectral coherence data from an electrode near an eye.

6. The method of claim 5, wherein the eye blink artifact is described using a slow delta band in a range of 0.5-1.0 Hz from one or more electrode positions.

7. The method of claim 1, wherein the orthogonalized spectral coherence data reduces a dimensionality of the smoothed spectral coherence data.

8. The method of claim 1, further comprising performing a jackknifing procedure on the orthogonalized spectral coherence data to generate a cross-validated result for the plurality of diagnostic rules and the diagnosis.

9. The method of claim 8, further comprising performing a multiple split half replication on the orthogonalized spectral coherence data to evaluate classification success of the plurality of diagnostic rules and the diagnosis.

10. The method of claim 9, further comprising format-converting a refined electroencephalograph (EEG) data to generate the Laplacian formatted electroencephalograph (EEG) data.

11. The method of claim 10, wherein the Laplacian formatted electroencephalograph (EEG) data is a scalp current potential data.

12. The method of claim 10, further comprising removing a non-muscle-induced artifact from a filtered electroencephalograph (EEG) data to generate the refined electroencephalograph (EEG) data.

13. The method of claim 12, wherein the refined EEG data is generated using source analysis.

14. The method of claim 12, further comprising removing a muscle-induced artifact from an initially processed electroencephalograph (EEG) data to generate the filtered electroencephalograph (EEG) data.

15. The method of claim 14, further comprising removing a gross artifact in a raw electroencephalograph (EEG) data to generate the initially processed electroencephalograph (EEG) data.

16. The method of claim 15, further comprising collecting the raw electroencephalograph (EEG) data from a subject population.

17. An apparatus comprising:
means for performing a spectral analysis on a Laplacian formatted electroencephalograph (EEG) data to generate a spectral coherence data;
means for performing a plurality of regression analysis on the spectral coherence data to generate a smoothed spectral coherence data;
means for performing a principal component analysis (PCA) on the smoothed spectral coherence data to generate an orthogonalized spectral coherence data; and
means for performing a multivariate discriminant function analysis (DFA) on the orthogonalized spectral coherence data to generate a plurality of diagnostic rules and a diagnosis.

18. The apparatus of claim 17, further comprising:
means for performing a jackknifing procedure on the orthogonalized spectral coherence data to generate a cross-validated result for the plurality of diagnostic rules and the diagnosis; and
means for performing a multiple split half replication on the orthogonalized spectral coherence data to evaluate classification success of the plurality of diagnostic rules and the diagnosis.

19. The apparatus of claim 18, further comprising:
means for format-converting a refined electroencephalograph (EEG) data to generate the Laplacian formatted electroencephalograph (EEG) data;
means for removing a non-muscle-induced artifact from a filtered electroencephalograph (EEG) data to generate the refined electroencephalograph (EEG) data; and
means for removing a muscle-induced artifact from an initially processed electroencephalograph (EEG) data to generate the filtered electroencephalograph (EEG) data.

20. The apparatus of claim 19, further comprising:
means for removing a gross artifact in a raw electroencephalograph (EEG) data to generate the initially processed electroencephalograph (EEG) data; and
means for collecting the raw electroencephalograph (EEG) data from a subject population.
